(19) United States
(12) Patent Application Publication
(10) Pub. No.: US 2011/0288298 A1
(43) Pub. Date: Nov. 24, 2011

(54) NOVEL POLYMORPH OF EMTRICITABINE AND A PROCESS FOR PREPARING OF THE SAME

(75) Inventors: Girij Pal Singh, Maharashtra (IN); Dhananjai Srivastava, Maharashtra (IN); Harishchandra Jadhav, Maharashtra (IN); Shailendra Pathak, Maharashtra (IN); Manmeet Saini, Maharashtra (IN); Sudhakar Patil, Maharashtra (IN)

(73) Assignee: LUPIN LIMITED, Mumbai (IN)

(21) Appl. No.: 13/128,604

(22) PCT Filed: Nov. 12, 2008
(86) PCT No.: PCT/IN08/00767

§ 371 Date: Jul. 26, 2011

(51) Int. Cl. C07D 411/04

(52) U.S. Cl. 544/317

(57) ABSTRACT

A polymorph of emtricitabine, wherein said polymorph displays angular positions of characteristic peaks in powder X-ray diffraction pattern 13.61±0.2, 15.54±0.2, 19.49±0.2, 20.55±0.2, 25.89±0.2, 28.09±0.2 and 29.10±0.2. A pharmaceutical composition comprising a polymorph of emtricitabine displaying angular positions of characteristic peaks in powder X-ray diffraction pattern 13.61±0.2, 15.54±0.2, 19.49±0.2, 20.55±0.2, 25.89±0.2, 28.09±0.2 and 29.10±0.2. A process for the preparation of a polymorph of emtricitabine comprising the steps of (a) dissolving crude emtricitabine in polar organic solvent by heating at a temperature of at least 40° C. and not more than 150° C. to form a reaction mixture optionally decreasing the concentration of polar organic solvent in said reaction mixture; cooling the reaction mixture obtained in step (a); and separating the solid from the cooled reaction mixture resulted in step (b).

### FIELD OF THE INVENTION

The present invention relates to a novel polymorph of emtricitabine and a process for preparing the same.

### BACKGROUND OF THE INVENTION

Emtricitabine and their derivatives are useful in the treatment of anti-viral diseases including HIV viral diseases and HAB viral diseases. Emtricitabine is the (−)-enantiomer of 4-amino-5-fluoro-1-[(2R,5S)-2-(hydroxymethyl)-1,3-oxathiolan-5-yl]-2(1H)-pyrimidinone, which is marketed in the name of Emritiva by Glaxosmithkline Beecham in US.

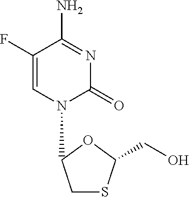

The first disclosure of the emtricitabine, which is also designated as (−)-cis FTC is found in the U.S. Pat. No. 6,624,245 and U.S. Pat. No. 6,703,396. The U.S. Pat. No. '396 describes the preparation of the emtricitabine from the reaction mixture containing emtricitabine in methanol by rotary evaporation followed by thin layer chromatography using a mobile phase ethylacetate:methanol (5:1). The U.S. Pat. No. 5,538,975 describes the purification of emtricitabine from column chromatography using methanol:ethylacetate as the eluent.

It is well known in the state of art that the crystalline form of a pharmaceutical substance affect the dissolution rate, solubility and bioavailability. Pharmaceutical active agents often exist in two or more crystalline forms that have different key physical and pharmaceutical properties including hygroscopicity, solubility, storage stability, density, hardness, flow properties and bioavailability. The crystalline form may be controlled by process employed for the manufacture of the pharmaceutical substance. In particular, the process of purification of the solid substance by crystallization is used to control the solid form (Organic Process Research & Development 2003, 7, 958-1027).

The U.S. Pat. No. 6,723,728 describes the different polymorphic forms form II and form III of emtricitabine which is distinctly different from the form I of emtricitabine, which is obtained by the process disclosed in the U.S. Pat. Nos. '396 and '245.

Form I of the emtricitabine is a crystalline form having characteristic peaks in powder X-ray diffraction pattern at 14.1, 19.9, 20.2, 20.6, 21.0, 22.4, 28.5, 29.5 and 32.6 and exhibiting a typical DSC (Differential Scanning calorimetry) thermogram with an onset of the peak at 151° C. and peak at 153.25° C. obtained by heating at rate of 10° C./minute.

Form II of the emtricitabine is a crystalline form having characteristic peaks in powder X-ray diffraction pattern at 14.7, 16.7, 19.6, 21.1, 21.8, 24.6 and 25.6; and Form III of the emtricitabine is a crystalline form having characteristic peaks in powder X-ray diffraction pattern at 14.5, 16.7, 19.6, 20.4, 21.4, 21.7, 25.2 and 26.2.

The U.S. Pat. No. '728 states that the form I and form III are the enantiotropic forms of form II. This patent reveals the transition of form I to form II by recrystallization after melting at 151° C. in example-1 and the formation of form III of the emtricitabine by heating form 1 to 160° C., that is just above the melting point of form I followed cooling to 25° C. in example-2. The form III does not show the endotherm formed at 151° C. as in form I. The endotherm at 162° C. is formed during the melting of form II emtricitabine; and the endotherm at 102° C. is formed during solid-state transition of form III emtricitabine to form II emtricitabine as described in the examples of the U.S. Pat. No. '728.

### SUMMARY OF THE INVENTION

The primary objective of the invention is to provide a novel polymorph of emtricitabine and a process for preparing the same.

It is an aspect of the present invention is to provide a polymorph of emtricitabine displays the following angular positions (two theta) of characteristic peaks in a powder X-ray diffraction pattern is 13.61±0.2, 15.54±0.2, 19.49±0.2, 20.55±0.2, 25.89±0.2, 28.09±0.2 and 29.10±0.2

It is another aspect of the present invention is to provide a polymorph of emtricitabine, which display the following angular positions (two theta) of characteristic peaks in a powder X-ray diffraction pattern is 13.61±0.2, 15.54±0.2, 19.49±0.2, 20.55±0.2, 25.89±0.2, 28.09±0.2 and 29.10±0.2 is essentially free from form II; and form III of emtricitabine.

It is yet another aspect of the present invention is to provide a pharmaceutical composition comprising a polymorph of emtricitabine, which display the following angular positions (two theta) of characteristic peaks in a powder X-ray diffraction pattern is 13.61±0.2, 15.54±0.2, 19.49±0.2, 20.55±0.2, 25.89±0.2, 28.09±0.2 and 29.10±0.2 is essentially free from form II; and/or form III of emtricitabine.

It is still another aspect of the present invention is to provide a pharmaceutical composition comprising emtricitabine with a HPLC purity of more than 98%, wherein said emtricitabine is a polymorph which displays the following angular positions of characteristic peaks in powder X-ray diffraction pattern 13.61±0.2, 15.54±0.2, 19.49±0.2, 20.55±0.2, 25.89±0.2, 28.09±0.2 and 29.10±0.2.

It is further an aspect of the invention to provide a process for the preparation of a polymorph of emtricitabine, which display the following angular positions (two theta) of characteristic peaks in a powder X-ray diffraction pattern is 13.61±0.2, 15.54±0.2, 19.49±0.2, 20.55±0.2, 25.89±0.2, 28.09±0.2 and 29.10±0.2.

### DESCRIPTION OF THE DRAWINGS

FIG. 1 is an X-ray powder diffraction pattern of a novel polymorph of emtricitabine of the present invention.

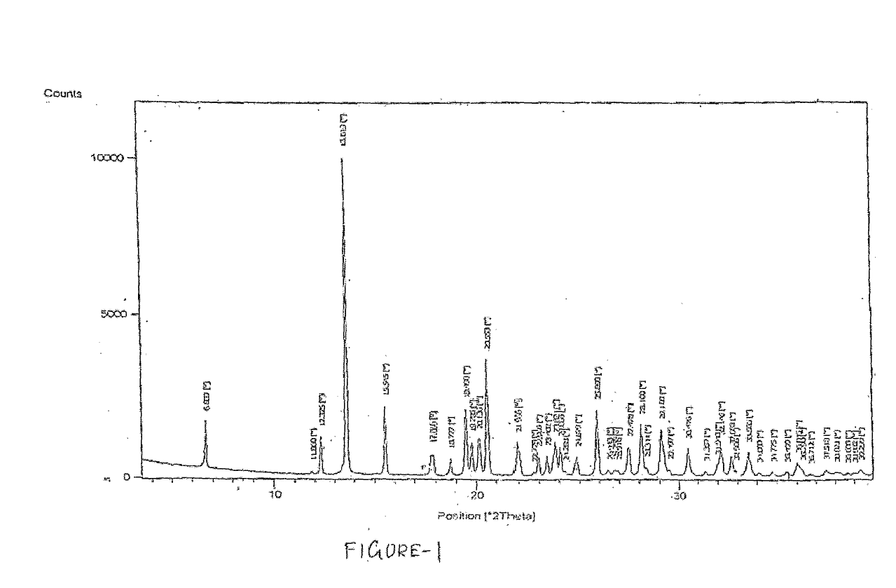

FIG. 2 is a DSC thermogram of a novel polymorph of emtricitabine by heating at 2° C. per minute of the present invention.

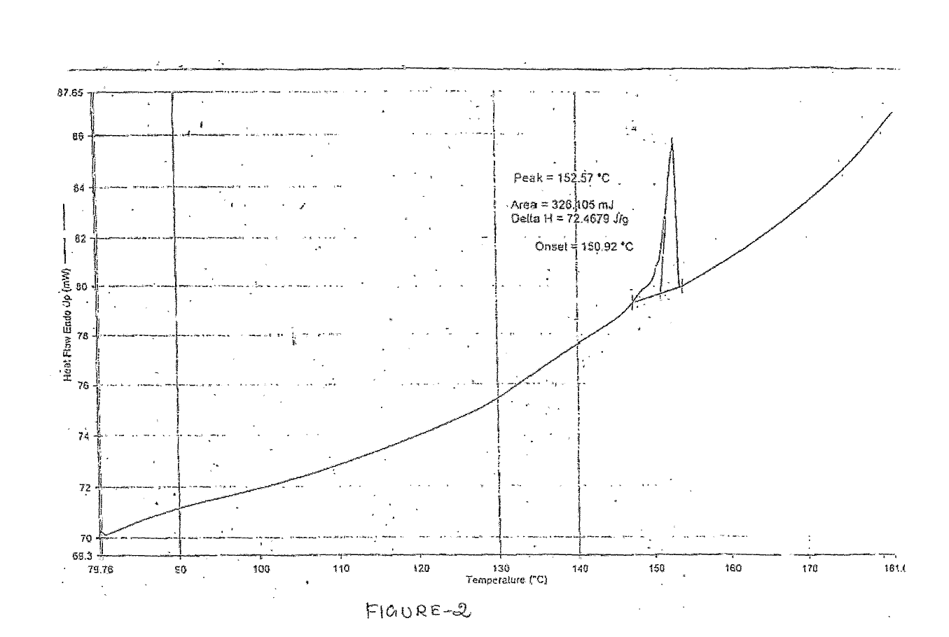

FIG. 3 is DSC thermogram of a novel polymorph of emtricitabine by heating at 5° C. per minute of the present invention.

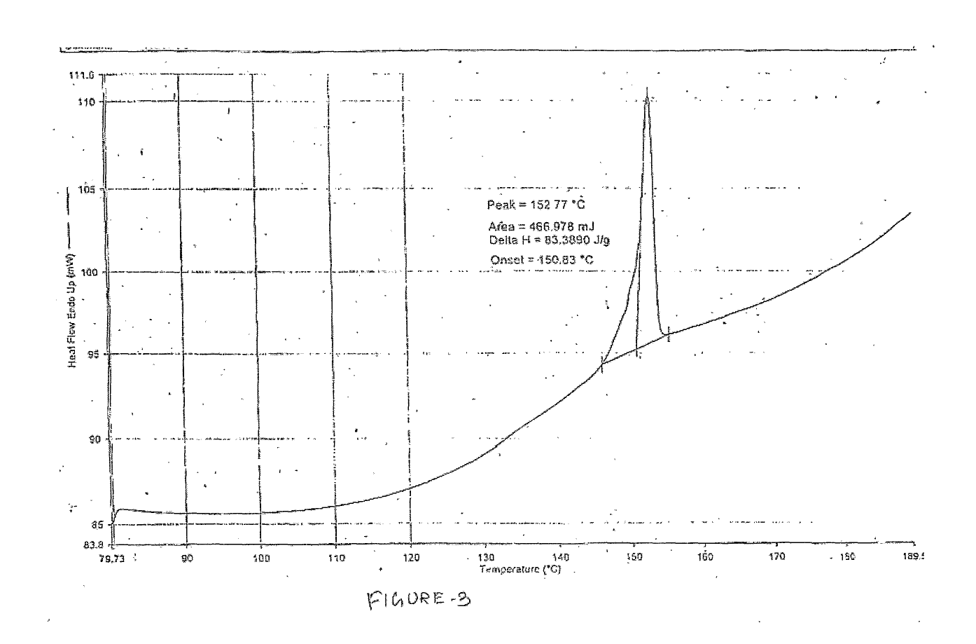

FIG. 4 is DSC thermogram of a novel polymorph of emtricitabine by heating at 10° C. per minute of the present invention.

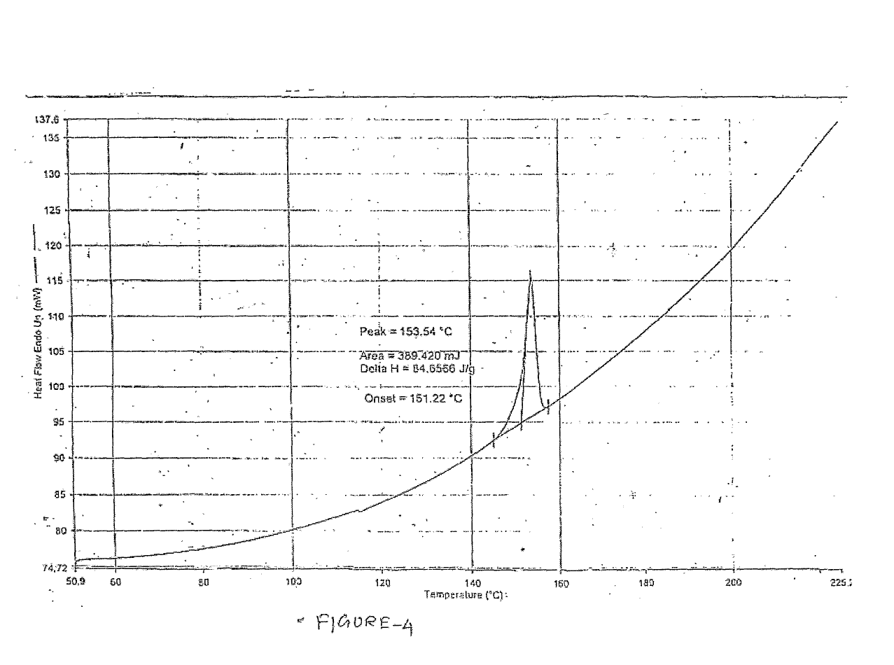

### DETAILED DESCRIPTION OF THE INVENTION

The inventors of the present invention have surprisingly found a novel polymorph of emtricitabine, which displays the following distinct angular positions (two theta) of characteristic peaks in a powder X-ray diffraction pattern is 13.61±0.2, 15.54±0.2, 19.49±0.2, 20.55±0.2, 25.89±0.2, 28.09±0.2 and 29.10±0.2, distinct from the reported polymorphs. The novel polymorph of emtricitabine displays DSC thermogram with an endotherm at 151° C. and no endotherms at about 102° C. or 162° C. obtained by heating at 2° C., 5° C. and 10° C. per minute.

The novel polymorph of emtricitabine display the following angular positions (two theta) of characteristic peaks in a powder X-ray diffraction pattern is 13.61±0.2, 15.54±0.2, 19.49±0.2, 20.55±0.2, 25.89±0.2, 28.09±0.2 and 29.10±0.2 is essentially free from form II; and form III of emtricitabine. The novel polymorph of emtricitabine essentially free from form II; and form III herein denotes that the novel polymorph of the present invention does not contain detectable amounts of form II and/or form III.

The form II and form III herein denotes the different polymorphic forms of emtricitabine as designated in the U.S. Pat. No. 6,723,728.

In an embodiment of the present invention, the novel polymorph of emtricitabine is prepared from crude emtricitabine involving the steps of:
(a) dissolving crude emtricitabine in a polar organic solvent by heating at a temperature of at least 40° C. and not more than 150° C. to form a reaction mixture; optionally decreasing the concentration of polar organic solvent in said reaction mixture;
(b) cooling the reaction mixture obtained in step (a); and
(c) separating the solid from the cooled reaction mixture resulted in step (b).

Crude emtricitabine herein denotes the (−)-cis form of 4-amino-5-fluoro-1-[(2R,5S)-2-(hydroxymethyl)-1,3-oxathiolan-5-yl]-2(1H)-pyrimidinone obtained by in any of the stereoselective synthesis methods or separation methods known to a person skilled in the art. Crude emtricitabine further includes the acid addition salts such as emtricitabine salicylic acid or it derivatives such as 5′-O-acyl derivatives or any such equivalent forms.

Said step of dissolving emtricitabine in the polar organic solvent is carried out in temperatures of at least 40° C. and not more than 150° C., preferably in the range of 45° C. to 100° C.

Said polar organic solvent may be a low carbon polar organic solvent. Preferred said polar organic solvents are alcohols such as methanol or ethanol or mixtures thereof. It should be realized that water may be present during the exposing of the polar organic solvent.

Said step of cooling the reaction mixture obtained in step (a) is carried out either by slowly cooling the said solution to 5±2° C. or by slowly cooling said solution to ambient temperature followed by cooling to 5±2° C. Ambient temperature herein denoted the temperature selected from the range 20° C. to 25° C.

The optional decreasing of the concentration of the polar organic solvent is carried out by distilling the excess of the polar organic solvent from said reaction mixture or by addition of the non-solvent or its mixtures such as isopropyl acetate or hexane to the reaction mixture. Non-solvent herein denotes the solvent that decreases the solubility of emtricitabine in the polar organic solvent.

The present invention is further illustrated by the following examples, which are provided merely to be exemplary of the invention and are not intended to limit the scope of the invention.

### Example: 1

### Preparation of Novel Polymorph of Emtricitabine

Crude emtricitabine (13 gm) was dissolved in ethanol 130 ml at 75° C. and charcolized. The reaction mixture was transferred to crystallization vessel and excess of solvent was distilled off to 40 ml. The reaction mixture was cooled to ambient temperature and then cooled further to 5° C., stirred for 2 hour at the same temperature. The resulting solid was filtered, washed with chilled ethanol and dried under reduced pressure to obtain emtricitabine (10.4 gm) of 99.5% purity by HPLC.

1. A polymorph of emtricitabine, wherein said polymorph displays the following angular positions of characteristic peaks in powder X-ray diffraction pattern 13.61±0.2, 15.54±0.2, 19.49±0.2, 20.55±0.2, 25.89±0.2, 28.09±0.2 and 29.10±0.2.

2. The polymorph of emtricitabine according to claim 1, wherein said polymorph is essentially free of form II and form III.

3. A pharmaceutical composition comprising a polymorph of emtricitabine, wherein said polymorph displays the following angular positions of characteristic peaks in powder X-ray diffraction pattern 13.61±0.2, 15.54±0.2, 19.49±0.2, 20.55±0.2, 25.89±0.2, 28.09±0.2 and 29.10±0.2.

4. The pharmaceutical composition according to claim 3, said polymorph is essentially free of form II and form III.

5. A pharmaceutical composition comprising emtricitabine with a HPLC purity of more than 98%, wherein said emtricitabine is a polymorph which displays the following angular positions of characteristic peaks in powder X-ray diffraction pattern 13.61±0.2, 15.54±0.2, 19.49±0.2, 20.55±0.2, 25.89±0.2, 28.09±0.2 and 29.10±0.2.

6. A process for the preparation of a polymorph of emtricitabine, wherein said process comprises the steps of:
(a) dissolving crude emtricitabine in polar organic solvent by heating at a temperature of at least 40° C. and not more than 150° C. to form a reaction mixture optionally decreasing the concentration of polar organic solvent in said reaction mixture;
(b) cooling the reaction mixture obtained in step (a); and
(c) separating the solid from the cooled reaction mixture resulted in step (b).

7. The process for the preparation of a polymorph of emtricitabine according to claim 6, wherein the polar organic solvent is selected from the group comprising alcohol and mixtures thereof.

8. The process for the preparation of a polymorph of emtricitabine according to claim 7, wherein the preferred polar organic solvent is ethanol or methanol.

9. The process for the preparation of a polymorph of emtricitabine according to claim 6, wherein said heating in step (a) is carried out preferably in the temperatures in the range of 40° C. to 100° C.
